(19) United States
(12) Patent Application Publication
(10) Pub. No.: US 2010/0203636 A1
(43) Pub. Date: Aug. 12, 2010

(54) Method for isolating islets of langerhans

(76) Inventor: Illani Atwater, Santa Barbara, CA (US)

Correspondence Address: FLOYD B. CAROTHERS;CAROTHERS AND CAROTHERS, 445 FORT PITT BOULEVARD, SUITE 200, PITTSBURGH, PA, 15219, US

(21) Appl. No.: 12/799,285

(22) Filed: Apr. 21, 2010

Related U.S. Application Data
(63) Continuation-in-part of application No. 11/291,549, filed on Dec. 1, 2005.

(51) Int. Cl. C12N 5/071

(52) U.S. Cl. 435/366

(57) ABSTRACT

Islets of Langerhans from a pancreas are isolated by exposing the pancreas to a very high-glucose (approximately 60 mM to 900 mM) solution to cause shrinking and accommodation of the acinar cells to the solution. Thereafter the very high-glucose solution is replaced with a low or zero-glucose solution to thereby selectively osmotically shock and swell acinar cells of the pancreas to destroy acinar tissue. If not previously minced during the initial exposure step, the treated pancreas is then minced, centrifuged and washed to thereby remove dead acinar cells and cell contents to leave islets of Langerhans. The islets are then placed in culture.

### CROSS REFERENCE

This application is a continuation-in-part of U.S. patent application Ser. No. 11/291,549, filed 12/01/2005, for Method for Isolation of Cells.

### FIELD OF THE INVENTION

The present invention relates to a method for the isolation of islets of Langerhans for transplantation.

### BACKGROUND OF THE INVENTION

Isolation of cells such as islets, neurons, incretin secreting cells and cells transfected with the GLUT 2 gene from other cells under known present day technologies provides disappointing results with low success rates. For example, isolation of islets of Langerhans under present day technology can at maximum yield only about half the islets available in pancreatic tissue. Furthermore, under existing procedures, many of the isolated islets are not viable.

Diabetes, hyperglycemia and impaired glucose tolerance are endocrine disorders characterized by inadequate production or use of insulin, which affects the metabolism of carbohydrates, proteins and lipids resulting in abnormal levels of glucose in the blood. Diabetes is a heterogeneous disease that can be classified into two major groups. One group is type I diabetes (also known as insulin-dependent diabetes, IDDM, juvenile diabetes, or auto-immune diabetes) and type II diabetes (non-insulin dependent diabetes, NIDDM, maturity-onset diabetes).

The cause of the raised glucose levels with insulin-dependent diabetes mellitus (IDDM) is the auto-immune destruction of pancreatic islets of Langerhans and, thus, insufficient secretion of the hormone insulin by the pancreas. In the absence of this hormone, the bodies' cells are not able to absorb glucose from the bloodstream causing an accumulation in the blood. Chronically elevated blood glucose damages tissues and organs. IDDM is normally treated with multiple daily insulin injections. The size and timing of insulin injections are determined by measurements of blood glucose and influenced by diet, exercise and stress.

Replenishment of functional glucose-sensing, insulin-secreting pancreatic beta cells through islet transplantation has been a long standing therapeutic target. Recently, a newly developed immune-suppression protocol has dramatically improved the survival of transplanted islets in auto-immune patients. Now the limiting factor in this approach is the availability of an islet source that is safe, reproducible, and abundant. Current methodologies use either human cadaverous material or porcine islets as transplant substrates. Significant problems encountered are the low availability of donor tissue, the variability and low yield of islets obtained via enzymatic dissociation and physical damage that may occur as a result of the isolation process. Separating islets of Langerhans, which represent only about 2% of the pancreatic volume, from the acinar portion of the pancreas is a difficult process. The relatively low success in this procedure has kept islet transplantation an experimental procedure of limited application.

A typical procedure for removal of islets from the pancreas to obtain a tissue suspension of islets and pancreatic tissue is to break down the pancreatic tissue with a digestive enzyme such as collagenase to free the islets. A difficulty with collagenase digestion of the pancreatic tissue to free the islets is that the individual islets are freed at different rates based on their size, distribution, and degree of entrainment or adherence in the tissue. Therefore, during the time collagenase is digesting pancreatic tissue to free the unfreed islets, it is also continuing to act on the islets that have already been freed, thereby breaking up the freed islets into small groups and even individual cells and degrading those cells. The result is that the number of viable islets that are freed by this process is much less than the number of islets in the pancreatic sample that is processed.

Once the islets are separated from the pancreas and suspended in a solution containing islets and partially digested pancreas tissue fragments, there are several techniques for concentrating and purifying them. The most common includes centrifuging the suspension and re-suspending the solution with one containing Ficoll or Percoll, and then centrifuging the tissue fragments through the density gradient so that the islets can be separated from the tissue fragments. Another technique for concentration and purification is the use of filtering, either alone or in combination with centrifuging. Another method partially concentrates the islets by using gravity sedimentation of islets through an inclined channel with a collection well at the bottom. The partially concentrated islets can then be further concentrated with a minimum of ordinary centrifugation or filtering or other processes known in the art.

The separation was first made possible by Lacy's development of a method for differential digestion by collagenase in rats, and this has remained the method used until today. For human islet isolation, a specially purified collagenase is used, Liberase, which is very expensive, costing over $2000 US for each isolation procedure. Ricordi modified the method for use in human pancreas tissue, which is enormously variable in texture, in order to maximize the number of islets separated from each pancreas. His method is known as the semi-automated method, and still requires a very expert team for successful isolation. The time of exposure to Liberase is different for each pancreas, and has to be monitored minute to minute. Nevertheless, only about half of the pancreases processed by an expert team yield enough islets to be transplanted, and, since many of the isolated islets are not viable, most patients require more than one islet preparation to be free of insulin injections.

### SUMMARY OF THE INVENTION

The method of the present invention utilizes differential selective osmotic shock for isolating islets for transplantation. The method is based upon the use of very high glucose (approximately 60 mM to 900 mM glucose) concentrations of solute followed by substitution with low or zero- glucose solute to differentially destroy non-selected acinar cells by osmotic shock. The method yields higher numbers of viable cells, the separation can be carried out in the cold, and the process is much faster and much cheaper than traditional methods. Furthermore, the procedure is a physical process and therefore does not depend upon size and texture of the individual pancreas. The method of the present invention can therefore be fully automated such as by using a machine in the situation for isolating islets of Langerhans wherein the machine is designed to receive the pancreas at one end and deliver the islets at the other.

The term glucose as used herein includes glucose and glucose substitutes. Thus, some non-metabolizable glucose substitutes which are also transported by GLUT 2 can also be used, such as 3-O-methyl-glucose or glucosamine. Also, mannuheptalose can be used to allow glucose to be transported but not metabolized in the islet cells. The method may be carried out in the cold or at room temperature, reducing warm ischemia time, and the osmotically destroyed cells are removed by gentle centrifugation and replacing the solution.

With particular reference to the isolation of islets of Langerhans from a pancreas, the method of the present invention comprises first mincing the pancreas, then exposing it to a very high-glucose (approximately 60 mM to 900 mM glucose) solution, and after allowing a period of time (approximately 3 to 60 minutes) for shrinking and accommodation of the acinar cells to the hypertonic solution in an approximate temperature range of 4° C. to 37° C., replacing the very high-glucose solution with a low or zero-glucose solution, thereby osmotically bursting the acinar cells of the pancreas. The treated pancreas tissue suspension is then centrifuged and washed to remove dead acinar cells and cell contents. The islets of Langerhans thus isolated are thereafter placed in culture.

The very high glucose solution may be slowly injected into the duct of the pancreas over a period of approximately five minutes. Alternatively, and preferably, the pancreas may be minced in the very high glucose solution. After injecting or mincing, it is further desirable to immerse the pancreas tissue in the same very high-glucose solution for a time period, such as approximately 3 to 60 minutes. A typical time period for thereafter immersing the pancreas in a low or zero-glucose solution is approximately fifteen minutes, or for a period of time sufficient to cause osmotic shock and bursting of the acinar cells. After the mincing and selective osmotic shocking, the pancreas may thereafter also be gently shaken in a cold solution, for example, for an approximate time of ten to fifteen minutes. Centrifuging and washing may be carried out multiple times, for example several times.

The isolated islets in culture may be maintained typially at 37° C. in a 5% CO2 incubator. It is also desirable to test the viability of an aliquot of the islets by culturing them in RPMI 1640 with 9 mM glucose supplemented with 15% of FBS, or in Hank's solutions supplemented with albumin, first in 5 mm glucose, then in 15 mm glucose to stimulate secretion.

The solutions used are typically Hank's solutions, Krebs' solutions and physiological solutions.

### DETAILED DESCRIPTION OF PREFERRED EMBODIMENTS

In one example, the pancreas was extracted from the donor (anesthetized pig or human cadaveric donor) in the traditional fashion, after being perfused with cold preservation solution. It wass transported to the islet isolation laboratory in cold solution and cleaned of adhering tissues and fat. The duct was canulated and a modified very high-glucose (approximately 60 mM to 900 mM glucose) Hank's solution was injected slowly, over a period of approximately five minutes, to inflate the acinar portion of the pancreas. The pancreas was then left for approximately 3 to 60 minutes, or for a time period sufficient to cause shrinking and accommodation of the acinar cells to the solution immersed in the same very high-glucose solution on ice, or at room temperature. Then, the solution was replaced with a zero-glucose Hank's solution for a further period of approximately fifteen minutes, or for a time period sufficient to osmotically shock and swell acinar cells. The pancreas was minced and after a gentle shaking period of approximately ten to fifteen minutes in the cold, the mixture was centrifuged and washed several times to remove the dead acinar cells and cell contents. Finally, the islets were placed in culture typically at 37° C. in a 5% CO₂ incubator until transplant.

To test for viability, islets were cultured in RPMI 1640 medium, or another appropriate culture medium, with approximately 9 mM glucose and supplemented with 15% FBS or human albumin. Insulin secretion was measured in aliquots of the culture medium every 24 hours. After one and after seven days, the islets are tested for response to a glucose challenge. The medium was replaced for one with low glucose (3 mM) for two hours, an aliquot taken and then replaced again for one with 20 mM glucose. After two hours, a second aliquot was taken and the islets were returned to the usual culture medium. Aliquots were frozen until measured for insulin, in duplicate, by the Imulite elisa system. By comparing the two aliquots, secretion in response to glucose can be assessed and the viability of the islets can be determined.

The zero-glucose Hank's solution, in mM, was 120 NaCl, 4 KCl, 25 NaHCO₃, 3.5 NaPO₄, 1.2 CaCl₂, 1.2 MgCl₂, 10 HEPES, pH 7.4 (37° C.) and 7.2 (4° C.). The very high-glucose Hank's solution is the same as above with approximately 60 mM to 900 mM glucose added. Any physiological solution with and without very high concentrations of glucose may be used.

In the cold, glucose metabolism is severely inhibited in the islet cells. At room temperature, the cells may be protected by addition of mannoheptulose, a selective inhibitor of glucose phosphorylation (first step in glucose metabolism). Thus the glucose would be allowed to accumulate more quickly in the cytoplasm to balance the glucose concentration in the medium.

Other applications of the method of the present invention are described and discussed hereinafter.

In another procedure, young Landrace swine of 26-35 kg underwent total pancreatectomy under general anesthesia, without perfusion using a cold preservation solution. Immediately after extraction, the pancreas which was cleaned under sterile conditions, was weighed, submerged in povidone-iodine solution, and washed with 2 L of sterile saline solution, all at room temperature (approximately 18° C.).

Under a sterile laminar flow hood, the pancreas was chopped finely and divided into sterile 40-mL conical tubes, according to the experimental protocol. After weighing the content, the islets wre isolated by enzymatic digestion with Liberase CI (Roche Cat. No. 11814435001, Basel, Switzerland) for 20 to 23 minutes at 37° C. The recovery rate was more than 60% of the islets as free islets. Islets isolated by selective osmotic shock (SOS) were obtained as follows: samples of chopped tissue were exposed to RPMI 1640 media supplemented with either 300 mM of glucose or 600 mM of glucose for 20 or 40 minutes at room temperature (approximately 22° C.). All samples were then centrifuged for 2 minutes at 1000 rpm and the solution rapidly replaced by a zero-glucose RPMI medium. The tissue was gently mixed, centrifuged, and washed again with fresh zero-glucose medium; the washing procedure was again repeated. The resultant mixture was either placed in culture or further purified as follows. Each of the portions of tissue were then processed by gentle mechanical disruption by suction and expulsion from a sterile syringe without a needle for approximately 15 minutes. The resulting mixture was centrifuged through a density gradient of Histopaque 1077 (Sigma-Aldrich no. 1077, St. Louis, Mo.). The islet-enriched fraction was placed in RPMI 1640 culture media without bicarbonate, but with 5.6 mM glucose, 20 mM HEPES, 1% of streptomycin, 1% of penicillin, and 20% of inactivated horse serum at 37° C. in a 5% CO₂ incubator. The culture media were replaced the first day after isolation and thereafter every 48 hours. The same purification and culture conditions were used for the Liberase-treated samples.

To evaluate islet recovery after isolation, the total number of islets larger than 100 μm in diameter within an aliquot of 50 μL, were counted and multiplied it by the total volume. The procedure was performed on day 4 of culture after purification to evaluate the number of intact islets. The number of islets per gram of pancreatic tissue was also calculated.

Islet viability and function were evaluated using insulin secretion tests on day 4 after the isolation. Washed islets isolated under various experimental conditions were exposed to RPMI 1640 culture media with 5.6 mM glucose and 20% horse serum for 90 minutes at 37° C. in a 5% CO₂ atmosphere. Then the samples were centrifuged at 2000 rpm for 15 minutes and the supemates were collected and frozen at −20° C. The islets were resuspended in media as above but with 16.7 mM glucose and cultured for a further 90 minutes. The supemate was analyzed using an immulite/immunlite 1000 insulin determination assay (Diagnostic Products Corporation, Los Angeles, Calif.) with results expressed as μIU/mL. Finally, insulin secretion was normalized to basal secretion values and also to the number of islets in each dish.

After this selective osmotic shock (SOS) treatment, the number of isolated islets was highest using 600 mM glucose for 20 minutes, as counted after 4 days in culture. In a representative experiment, 4089 islets per gram of tissue were isolated from the portion of pancreas exposed to a 300 mM glucose solution, whereas 13,423 islets per gram of tissue were obtained from the portion exposed to 600 mM glucose. From the same pancreas, Liberase isolation yielded 8543 islets pr gram of tissue.

Insulin secretion from islets isolated by SOS was evaluated in response to a culture medium supplemented with 5.6 mM glucose or 16.7 mM glucose after 4 days in culture.

After 4 days in culture, islets responded to a physiological glucose stimulus showing an increment in insulin secretion of about 3-fold over basal values.

Islets isolated by exposure to very high glucose for 40 minutes showed lower levels of basal insulin secretion and responded poorly to glucose (results not shown). Islets isolated with Liberase showed no detectable insulin secretion, although the number of purified islets was similar.

Osmosis is the flow of solvent through a semi-permeable membrane to equalize the concentration of solutes on either side. In the case of living cells, water is the solvent. Solutes can be ions, sugars, amino acids, or other permeant particles. Thus, this process can be used to separate any type of cell from others, if the cell membrane is selectively permeable to some solute. In the case of the islet cells, the membrane contains a glucose transporter, GLUT 2, which rapidly (within seconds) transports glucose across the membrane to equalize the concentration on either side, thus avoiding a major movement of water. Most cells in the body are relatively impermeable to glucose, except in the presence of insulin (they have glucose transporters, GLUT 4, that are inserted into the membrane after activation of the insulin receptor). Thus, upon application of very high glucose, the other cells shrink rapidly. Most cells have many adaptation methods for osmotic shock (Na—Cl exchange, for example), and so over the next 15 minutes or so, the cells gradually return to their normal volume. When they are then exposed to the low or zero-glucose solution, the reverse occurs and the cells swell. If the shock is sufficient, they burst. In most cases, the osmotic shock is lethal, if not immediately, certainly eventually. Any remaining cells die in culture within 24 hours.

The SOS technique to isolate islets of Langerhans represents a novel approach in the field of islet cell transplantation. The elimination of enzymes from the islet isolation procedure has several advantages: enzymatic damage to islets is avoided, preparation purity is improved, and processing can be performed at cooler temperatures, is less costly and, most importantly, has the potential for full automation.

I claim:

1. A method for isolating islets of Langerhans from a pancreas, said method comprising:
exposing the pancreas to a very high-glucose, approximately 60 mM to 900 mM, solution to cause shrinking and accommodation of the acinar cells to the solution,
thereafter replacing the very high-glucose solution with a low or zero-glucose solution to thereby osmotically shock and swell acinar cells of the pancreas to destroy acinar tissue;
mincing the treated pancreas;
centrifuging and washing the minced pancreas to thereby remove dead acinar cells and cell contents to leave islets of Langerhans; and
placing the islets in culture.

2. The method of claim 1, wherein the step of mincing is carried out during the step of exposing, wherein the minced pancreas is immersed in said very high-glucose solution.

3. The method of claim 1, wherein the step of exposing includes slowly injecting the solution into the duct of the pancreas.

4. The method of claim 3, wherein the step of injecting is carried out over a period of approximately five minutes.

5. The method of claim 3, after the step of injecting, immersing the pancreas in the same very high-glucose solution.

6. The method of claim 1, wherein said pancreas is exposed to said very high-glucose solution for approximately 3 to 60 minutes.

7. The method of claim 1, wherein said step of exposing is carried out in a temperature range of approximately 4° C. to 37° C.

8. The method of claim 1, wherein after the step of replacing the high-glucose solution with low or zero-glucose solution, exposure of the cells to the low or zero-glucose is carried out for a period of approximately fifteen minutes.

9. The method of claim 1, wherein mincing is carried out after the step of exposing, and after the step of mincing the pancreas, applying a gentle shaking to the minced pancreas in applied cold.

10. The method of claim 9, wherein the step of gentle shaking is carried out for a period of approximately ten to fifteen minutes.

11. The method of claim 1, wherein the step of centrifuging and washing is carried out multiple times.

12. The method of claim 1, wherein the culture is maintained at approximately 37° C. in a 5% CO2 incubator.
